(19) United States
(12) Patent Application Publication
(10) Pub. No.: US 2018/0238789 A1
(43) Pub. Date: Aug. 23, 2018

(54) CORRELATION-BASED DETERMINATION OF PARTICLE CONCENTRATION FIELD

(71) Applicant: International Business Machines Corporation, Armonk, NY (US)

(72) Inventors: Yu Tao Ba, Beijing (CN); Wen Chen Cheng, Beijing (CN); Xin Jie Lv, Beijing (CN); Ling Yun Wang, Beijing (CN); Wen Jun Yin, Beijing (CN); Gang Zhou, Beijing (CN); Ke Xu Zou, Beijing (CN)

(21) Appl. No.: 15/436,161

(22) Filed: Feb. 17, 2017

(51) Int. Cl. G01N 15/06; G01N 33/00

(52) U.S. Cl. CPC G01N 15/06; G01N 2015/0046; G01N 33/0008

(57) ABSTRACT

A particle concentration is determined by acquiring a particle concentration measurement at a reference station from each of a plurality of sensors to be evaluated. A reference particulate concentration measurement is acquired from a reference station sensor at the reference station. A base set of sensors is selected from the plurality of sensors based upon a correlation calculation. A reference sensor is selected from the base set of sensors. A model is constructed that specifies a relationship between the reference sensor and the reference station sensor. A generalized sensor type is formulated using one or more characteristics of the reference sensor. The generalized sensor type is used to construct an inheritance network model. A particle concentration for the generalized sensor type is determined based upon the inheritance network model.

### FIELD

The present invention relates to determining airborne particle concentration and, in particular, to a correlation-based determination of airborne particulate concentration.

### BACKGROUND

Airborne particulates constitute a major component of urban air pollution. Anthropogenic sources include combustion within car engines, solid-fuel combustion in households, industrial activities, smelting, quarrying, mining, building, and manufacturing of cement, ceramics and bricks. Epidemiological evidence indicates a clear relationship between exposure to particulate matter and effects on health, particularly smaller particles that can reach the deep regions of the lungs.

With particulate matter, the size of the particles determines how long particles remain airborne to be inhaled, and whether the particles reach the deep regions of the lungs where they can be absorbed and potentially cause serious health problems. Thus, it is useful to define particle size criteria when sampling for airborne particulates.

When sampling for pollutants, two fractions are commonly used—Particulate Matter 10 (PM10) and Particulate Matter 2.5 (PM2.5). PM10 is a standard-size fraction of particles that are 10 microns or less in diameter. PM2.5 is a standard-size fraction of particles that are 2.5 microns or less in diameter.

Conventional approaches to performing PM2.5 concentration monitoring include manual sampling and automated sampling. Manual samplers draw a known volume of air through a filter. The filter is weighed on an analytical balance before and after sampling. Any difference in weight divided by the known volume of air pulled through the filter gives the mass concentration of the particulate. Two types of automated samplers in common usage include: samplers that use a beta gauge and beta attenuation monitoring (BAM) for mass measurement, and samplers that use a tapered element oscillating microbalance (TEOM) for mass measurement. The TEOM and BAM approaches are relatively expensive and include strict deployment requirements.

The TEOM technique operates by drawing air through a filter attached at the tip of a glass tube. An electrical circuit places the glass tube into oscillation. The resonant frequency of the tube is proportional to the square root of the mass in the filter.

The BAM monitoring technique exploits an absorption of beta radiation by solid particles extracted from air flow and can also detect PM10. In the BAM monitoring technique, air is pulled through a filter tape to accumulate a sample. The mass of the tape before and after sampling is determined by advancing the tape spot into a beta attenuation cell.

Yet another (less expensive) air monitoring technique uses light scattering methods and a micro-electromechanical systems (MEMS) particle sampler. These approaches use a coefficient calibration procedure before the particle sampler leaves the factory. Thereafter, the coefficient calibration procedure must be manually performed on a periodic basis. Particle mass is calculated using linear regression procedures, which may or may not incorporate corrections for relative humidity.

### SUMMARY

The following summary is merely intended to be exemplary. The summary is not intended to limit the scope of the claims.

A computer-implemented method for determining a particle concentration, in one aspect, may comprise acquiring a respective particulate concentration measurement at a reference station from each of a corresponding plurality of sensors to be evaluated, acquiring a reference particulate concentration measurement from a reference station sensor at the reference station, selecting a base set of sensors from the plurality of sensors based upon at least one correlation calculation between one or more of the plurality of sensors and the reference station sensor, selecting a reference sensor from the base set of sensors, constructing a particle concentration calculation model that specifies a relationship between the reference sensor and the reference station sensor, formulating a generalized sensor type using one or more characteristics of the reference sensor, using the generalized sensor type to construct an inheritance network model, and determining a particle concentration for the generalized sensor type based upon the inheritance network model.

Other aspects comprise a computer program product for determining a particle concentration and an apparatus for determining a particle concentration.

### BRIEF DESCRIPTION OF THE SEVERAL VIEWS OF THE DRAWINGS

The foregoing aspects and other features are explained in the following description, taken in connection with the accompanying drawings, wherein:

FIG. 1 illustrates a first exemplary computer-implemented method for determining a particle concentration in accordance with one or more embodiments of the present invention.

FIG. 2 illustrates an exemplary computer-implemented method for selecting a base set of sensors in accordance with the exemplary method of FIG. 1.

FIG. 3 illustrates an exemplary computer-implemented method for selecting a reference sensor from the base set of sensors in accordance with the exemplary method of FIG. 1.

FIG. 4 illustrates an exemplary error list for a plurality of sensors using a plurality of particle concentration models.

FIG. 5 illustrates an exemplary computer-implemented method for generating a particle concentration calculation model in accordance with the exemplary method of FIG. 1.

FIG. 6 illustrates an exemplary system in accordance with the exemplary method of FIG. 5.

FIG. 7 illustrates an exemplary inheritance network model for use with the method of FIG. 1. FIG. 8 illustrates an exemplary computer-implemented method for constructing an inheritance network model in accordance with the exemplary method of FIG. 1.

FIG. 9 illustrates an exemplary radar similarity chart in accordance with the exemplary method of FIG. 8.

FIG. 10 illustrates an exemplary apparatus in accordance with one or more embodiments of the present invention.

FIG. 11 depicts a cloud computing environment, according to embodiments of the present disclosure; and

FIG. 12 depicts abstraction model layers, according to embodiments of the present disclosure.

### DETAILED DESCRIPTION

For purposes of measuring PM2.5 and PM10 particle concentrations, the U.S. Environmental Protection Agency (EPA) defines a tier-based approach that determines how accurately an instrument under evaluation performs in comparison to a Federal Reference Method (FRM) or a Federal Equivalent Method (FEM). The FRM and FEM procedures are performed at specially designated reference sites or reference stations and provide a monitoring accuracy of 10% or better. To complement these FRM and FEM stations, supplemental monitoring and source identification monitoring tiers have been defined to have errors less than 20% and 30%, respectively.

The FRM and FEM procedures for measuring ambient concentrations of specified air pollutants have been designated as “reference methods” or “equivalent methods,” respectively, in accordance with Title 40, Part 53 of the Code of Federal Regulations (40 CFR Part 53). Subject to any limitations (e.g., operating range or temperature range) specified in the applicable designation, each method is acceptable for use in state or local air quality surveillance systems under 40 CFR Part 53 and 40 CFR Part 58. The European Union has similarly defined PM monitoring data quality as highlighted in a 2008/50/EC directive for both Reference and Indicative measurements, with total uncertainties of 25% and 50%, respectively.

The EPA has designated any of a multiplicity of reference methods as being suitable for measuring PM2.5 particle concentrations. Further information concerning these reference methods may be found in the Federal Register notices cited hereinafter for each illustrative method, or by contacting the National Exposure Research Laboratory, Exposure Methods and Measurement Division (MD-D205-03), U.S. Environmental Protection Agency, Research Triangle Park, N.C. 27711.

The following are illustrative examples of EPA-designated reference methods for measuring PM2.5 particle concentrations:

(1) Andersen Model RAAS2.5-200 PM2.5 Ambient Audit Air Sampler Manual Reference Method: RFPS-0299-128 “Andersen Instruments, Incorporated Model RAAS2.5-200 PM2.5 Audit Sampler,” configured as a PM2.5 reference method and operated with software (firmware) version 4B, 5.0.1-6.09, 6.0A, or 6.0B, for a continuous 24-hour sample period at a flow rate of 16.67 liters/minute, and in accordance with a corresponding Model RAAS2.5-200 Operator's Manual and with the requirements and sample collection filters specified in 40 CFR Part 50, Appendix L. Federal Register: Vol. 64, page 12167, Mar. 11, 1999.

(2) Graseby Andersen Model RAAS2.5-100 PM2.5 Ambient Air Sampler Manual Reference Method: RFPS-0598-119 “Graseby Andersen Model RAAS2.5-100 PM2.5 Ambient Air Sampler,” operated with software version 4B, 5.0.1-6.09, 6.0A, or 6.0B, configured for “Single 2.5” operation, for a continuous 24-hour sample period at a flow rate of 16.67 liters/minute, and in accordance with the Model RAAS2.5-100 Operator's Manual and with the requirements and sample collection filters specified in 40 CFR Part 50, Appendix L. Federal Register: Vol. 63, page 31991, Jun. 11, 1998.

(3) Met One Instruments, Inc. E-FRM-PM2.5 Manual Equivalent Method: EQPS-0316-235 “Met One Instruments, Inc. E-FRM,” configured for filter sampling of ambient particulate matter using the US EPA PM10 inlet specified in 40 CFR 50 Appendix L, FIGS. L-2 thru L-19, equipped with a URG-2000-30EGN Cyclone particle size separator, and operated for a continuous 24-hour sample period at a flow rate of 16.67 liters/minute, using 47 mm PTFE membrane filter media, and operating with firmware version R1.1.0 and later, and operated in accordance with the Met One EFRM PM2.5 operating manual. Federal Register: Vol. 81, page 25397, Apr. 28, 2016.

(4) Grimm Model EDM 180 PM2.5 Monitor Automated Equivalent Method: EQPM-0311-195 “Grimm Technologies, Inc. Model EDM 180 PM2.5 or Tisch Environmental TE-EDM 180 PM2.5 Monitor,” light scattering continuous ambient particulate monitor operated for 24 hours at a volumetric flow rate of 1.2 L/min, configured with a Nafion®-type air sample dryer, complete for operation with firmware version 7.80 or later, in accordance with the Grimm Technologies, Inc. Model EDM 180 Operation and Instruction Manual. The optional graphic presentation can be made with the software model 1.177 version 3.30 or later. Federal Register: Vol. 76, page 15974, Mar. 22, 2011 Latest Modification: January 2012; March 2014.

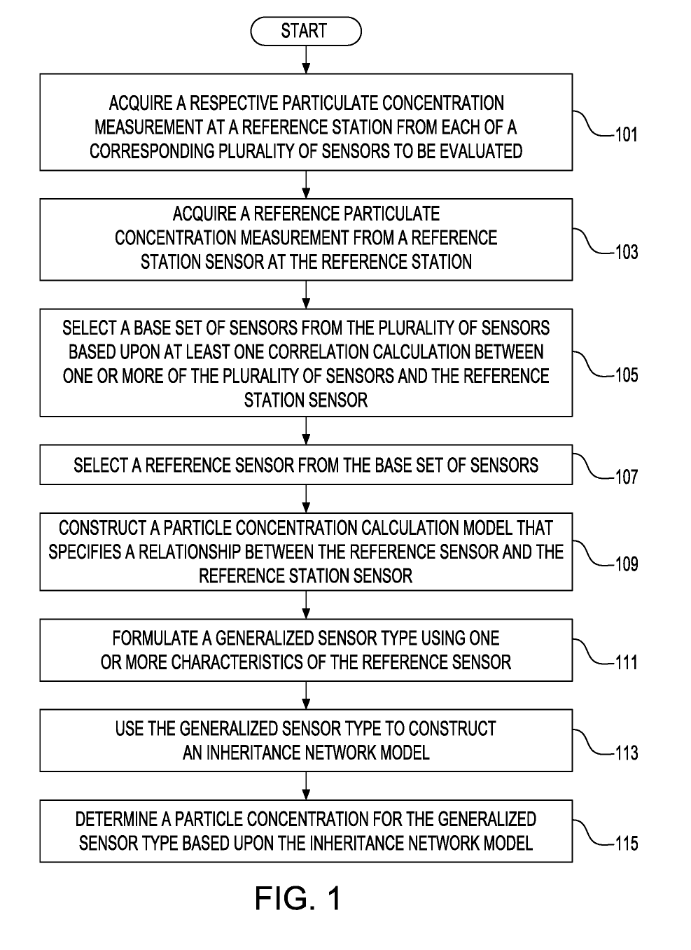

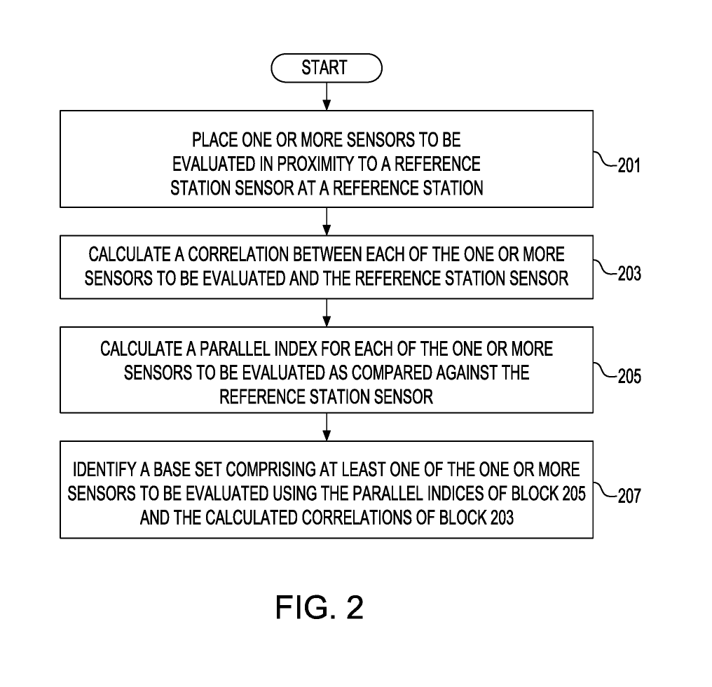

FIG. 1 illustrates a first exemplary computer-implemented method for determining a particle concentration in accordance with one or more embodiments of the present invention. The method commences at block 101 where a respective particulate concentration measurement is acquired at a reference station from each of a corresponding plurality of sensors to be evaluated. For purposes of illustration, the reference station may utilize the aforementioned EPA FRM and FEM procedures for measuring ambient concentrations of particulates. Next, at block 103, a reference particulate concentration measurement is acquired from a reference station sensor at the reference station. Then a base set of sensors is selected from the plurality of sensors based upon at least one correlation calculation between one or more of the plurality of sensors and the reference station sensor (block 105). This correlation calculation may be based upon uniformity or similarity. An illustrative procedure for selecting the base set of sensors is set forth hereinafter with reference to FIG. 2.

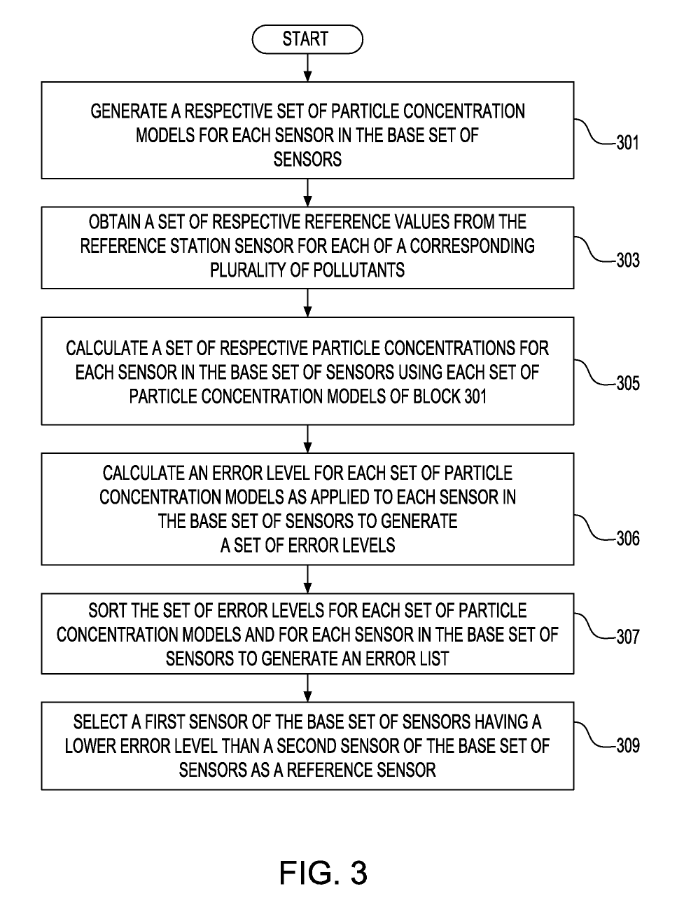

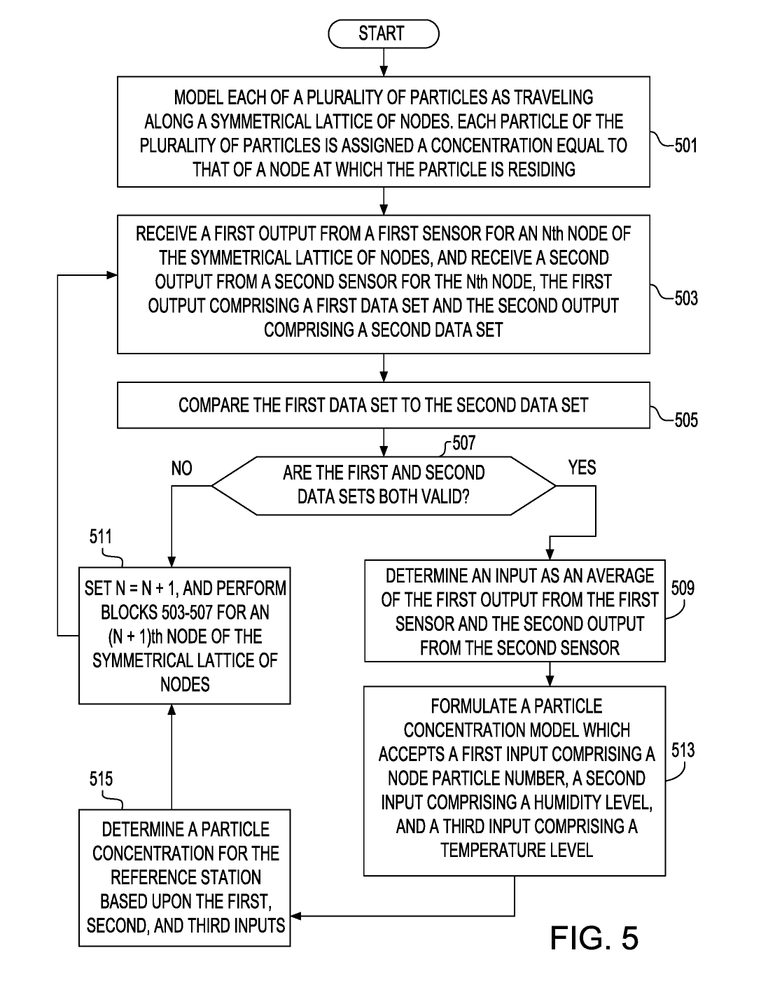

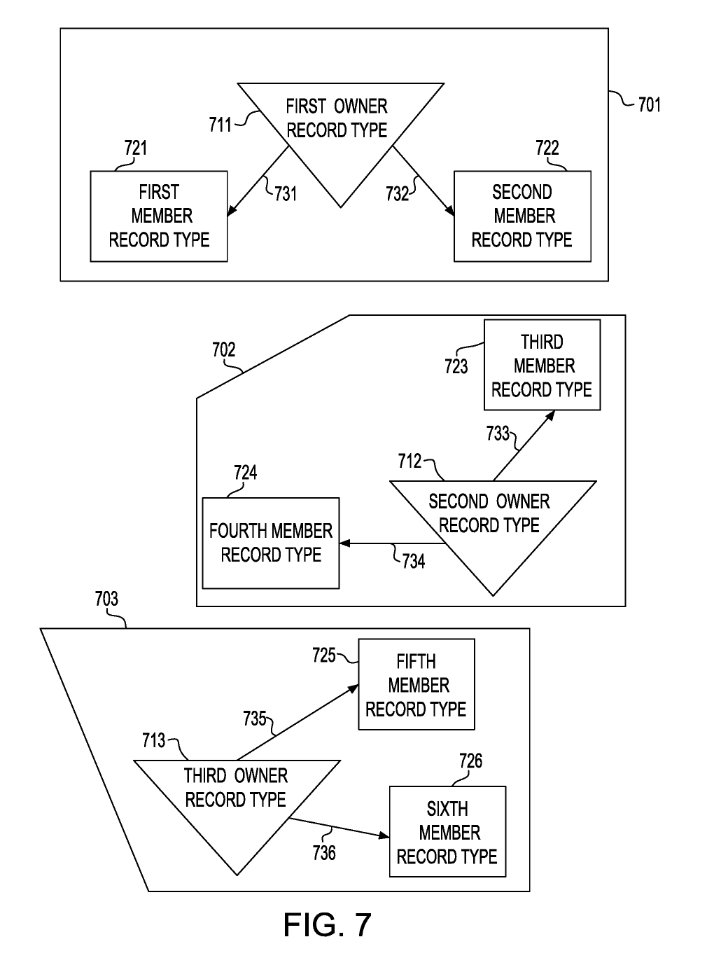

Returning to FIG. 1, the method advances to block 107 where a reference sensor is selected from the base set of sensors. An illustrative procedure for selecting the reference sensor from the base set of sensors is set forth hereinafter with reference to FIG. 3. Next, at block 109 (FIG. 1), a particle concentration calculation model is constructed that specifies a relationship between the reference sensor and the reference station sensor. FIG. 5 illustrates an exemplary computer-implemented method for generating the particle concentration calculation model. A generalized sensor type is formulated using one or more characteristics of the reference sensor (FIG. 1, block 111). This generalized sensor type is used to construct an inheritance network model (block 113). FIG. 8 illustrates an exemplary computer-implemented method for constructing the inheritance network model. Then, a particle concentration is determined for the generalized sensor type (FIG. 1, block 115) based upon the inheritance network model, as described in greater detail hereinafter with reference to FIGS. 7-9.

FIG. 2 illustrates an exemplary computer-implemented method for selecting a base set of sensors in accordance with the procedure of FIG. 1. The method commences at block 201 where one or more sensors to be evaluated are installed in proximity to a reference station sensor at a reference station. Then, at block 203, a correlation is calculated between each of the one or more sensors to be evaluated and the reference station sensor. A parallel index for each of the one or more sensors to be evaluated, as compared against the reference station sensor, is determined at block 205.

Parallel indexing is a technique that has been used in conjunction with radar navigation. This navigational technique involves creating a line on a radar screen that is parallel to a ship's course, but offset to the left or right by some distance. This parallel line allows the navigator to maintain a given distance away from hazards, shallow water, and shoreline features. In the present context, parallel indexing has been adapted for a very different and unique purpose. Parallel indexing is used, to plot one or more characteristics of each respective sensor to be evaluated against the same one or more characteristics of the reference station sensor. The plot is used to determine a level of similarity between each respective sensor to be evaluated and the reference station sensor, on the basis of each of the one or more characteristics.

At block 207, a base set comprising at least one of the one or more sensors to be evaluated is identified using the parallel index of block 205 and the calculated correlations of block 203. For purposes of illustration, the parallel index plot is used to identify a sensor, or a group of sensors, of the one or more sensors to be evaluated that is most similar to the reference station sensor. The calculated correlations are used to identify only those sensors of the sensors to be evaluated which provide at least an acceptable level of correlation.

FIG. 3 illustrates an exemplary computer-implemented method for selecting a reference sensor from the base set of sensors in accordance with the procedure of FIG. 1. The procedure commences at block 301 (FIG. 3) where a respective set of particle concentration models are generated for each sensor in the base set of sensors. Next, at block 303, a set of respective reference values are obtained from the reference station sensor for each of a corresponding plurality of pollutants. At block 305, a set of respective particle concentrations are calculated for each sensor in the base set of sensors using each set of particle concentration models of block 301. An error level is calculated for each set of particle concentration models as applied to each sensor in the base set of sensors to generate a set of error levels (block 306). At block 307, the set of error levels is sorted for each set of particle concentration models and for each sensor in the base set of sensors to generate an error list. Then, at block 309, a first sensor of the base set of sensors having a lower error than a second sensor of the base set of sensors is selected as a reference sensor.

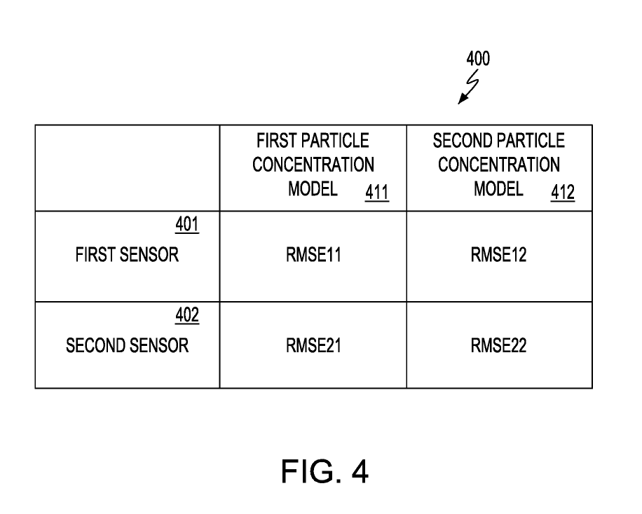

FIG. 4 illustrates an exemplary error list 400 for a plurality of sensors using a plurality of particle concentration models. A first root mean square error (RMSE11) is determined for a first particle concentration model 411 applied to a first sensor 401 of the base set of sensors. A second root mean square error (RMSE12) is determined for a second particle concentration model 412 applied to the first sensor 401. A third root mean square error (RMSE21) is determined for the first particle concentration model 411 applied to a second sensor 402 of the base set of sensors. A fourth root mean square error (RMSE22) is determined for the second particle concentration model 412 applied to the second sensor 402. Thus, the error list 400, mathematically denoted as EL₁, is equal to ΣRMSE (Sensorᵢ, L₁)/N. An error ranking (Errorrank) for sorting the error level at block 307 (FIG. 3) may be determined using Errorrank=EL1R+EL2R+EL3R+EL4R+EL5R+EL6R.

FIG. 5 illustrates an exemplary computer-implemented method for generating a particle concentration calculation model in accordance with the procedure of FIG. 1. The method commences at block 501 (FIG. 5) where a plurality of particles are each modeled as traveling along a symmetrical lattice of nodes. Each particle is assigned a concentration equal to that of a node at which the particle is residing. During each of a plurality of time steps, particles travel to new nodes in the symmetrical lattice, and the concentrations of those nodes is assigned to the concentration of the arriving particle. If more than one particle arrives at a node during a time step of the plurality of time steps, the resulting concentration at that node becomes the mean concentration of all particles arriving at the node. During a next step of the plurality of time steps, all particles carry the mean concentration to their next destination node.

The operational sequence of FIG. 5 progresses to block 503 where a first output from a first sensor for an Nth node of the symmetrical lattice of nodes is received. N is a positive integer greater than zero. A second output from a second sensor for the Nth node is also received. The first output comprises a first data set and the second output comprises a second data set. Then, at block 505, the first data set is compared to the second data set. At block 507, a test is performed to ascertain whether or not the first data set and the second data set are both valid. When a difference between the first data set and the second data set is equal to or less than a specified or a predetermined threshold, then the first and second data sets are both considered to be valid. Otherwise, the first and second data sets are considered to not be valid. The negative branch from block 507 leads to block 511 where N is set to N+1. The method then performs the steps of blocks 503-507 for an (N+1)th node of the symmetrical lattice of nodes.

The affirmative branch from block 507 leads to block 509 where an input is determined as an average of the first output from the first sensor and the second output from the second sensor. Then, at block 513, a particle concentration model is formulated which accepts a first input comprising a node particle number for the Nth node, a second input comprising a humidity level, and a third input comprising a temperature. Based on the first, second, and third inputs, the particle concentration model determines a particle concentration for the reference station (block 515). The method then loops back to block 511 (discussed previously).

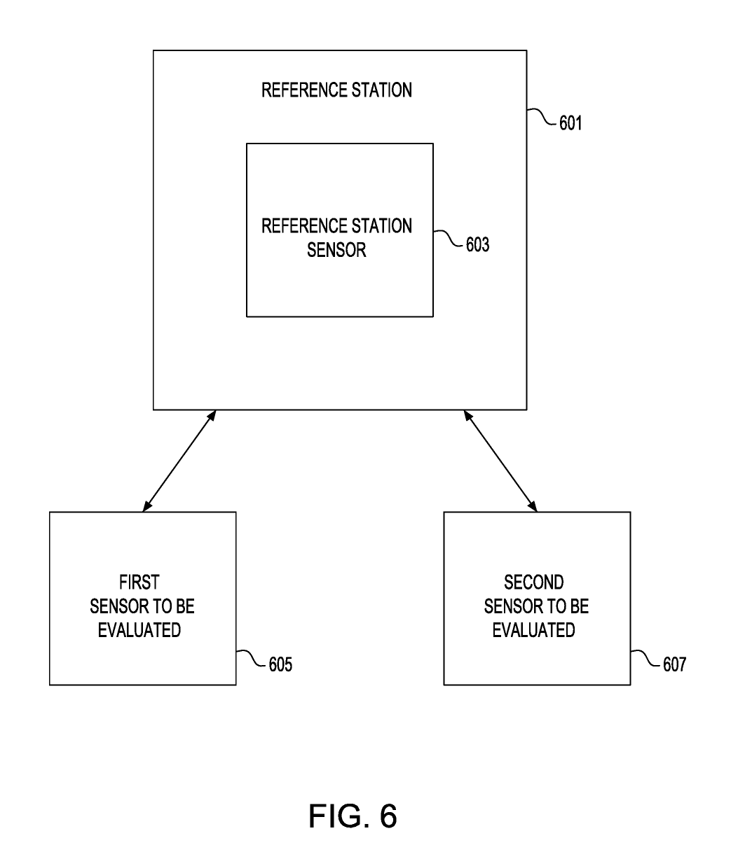

FIG. 6 illustrates an exemplary system for generating a particle concentration calculation model in accordance with the procedure of FIG. 5. A reference station 601 is equipped with a reference station sensor 603. For purposes of illustration, the reference station 601 may utilize any of the aforementioned EPA FRM and FEM procedures for measuring ambient concentrations of particulates. A first sensor 605 to be evaluated and a second sensor 607 to be evaluated are placed in proximity to the reference station sensor 603.

FIG. 7 illustrates an exemplary inheritance network model 700 for use with the method of FIG. 1. As used herein, the term “inheritance” refers to a concept where, when a class of objects is defined, any subclass that is defined can inherit the definitions of one or more general classes. An object in a subclass need not carry its own definition of data and methods that are generic to the class (or classes) of which it is a part. The concept of inheritance not only speeds up program development, but it also ensures an inherent validity to the defined subclass object. Data and methods that work and are consistent with regard to a class will also work for a subclass of the class.

Some types of data are naturally modeled using a network of nodes that has more than one parent node per child node. As used herein, the term “network model” refers to a network of nodes that permits a modeling of many-to-many relationships in data. The network model was defined by the Conference on Data Systems Languages (CODASYL) in 1971. The basic data modeling construct in the network model is the set construct. A complete network of relationships is represented using several pairwise sets, such as a first set 701, a second set 702, and a third set 703. A set may define a 1 to M relationship, M being a positive integer greater than one, although a 1 to 1 relationship is permitted. The network model is based upon mathematical set theory.

Each pairwise set comprises an owner record type and one or more member record types. For example, the first set 701 comprises a first owner record type 711, the second set 702 comprises a second owner record type 712, and the third set comprises a third owner record type 713. The first set 701 also comprises a first member record type 721 and a second member record type 722. Likewise, the second set 702 also comprises a third member record type 723 and a fourth member record type 724. Similarly, the third set 703 also comprises a fifth member record type 725 and a sixth member record type 726. The first owner record type 711 is indicated using a tail end of a first relationship arrow 731 and a second relationship arrow 732. The second owner record type 712 is indicated using a tail end of a third relationship arrow 733 and a fourth relationship arrow 734. The third owner record type 713 is indicated using a tail end of a fifth relationship arrow 735 and a tail end of a sixth relationship arrow 736.

The member record types 721, 722, 723, 724, 725 and 726 are indicated using a head end of a relationship arrow. For example, the first member record type 721 is indicated using a head end of the first relationship arrow 731. The second member record type 722 is indicated using a head end of the second relationship arrow 732. The third member record type 723 is indicated using a head end of the third relationship arrow 733. The fourth member record type 724 is indicated using a head end of the fourth relationship arrow 734. The fifth member record type 725 is indicated using a head end of the fifth relationship arrow 735. The sixth member record type 726 is indicated using a head end of the sixth relationship arrow 736.

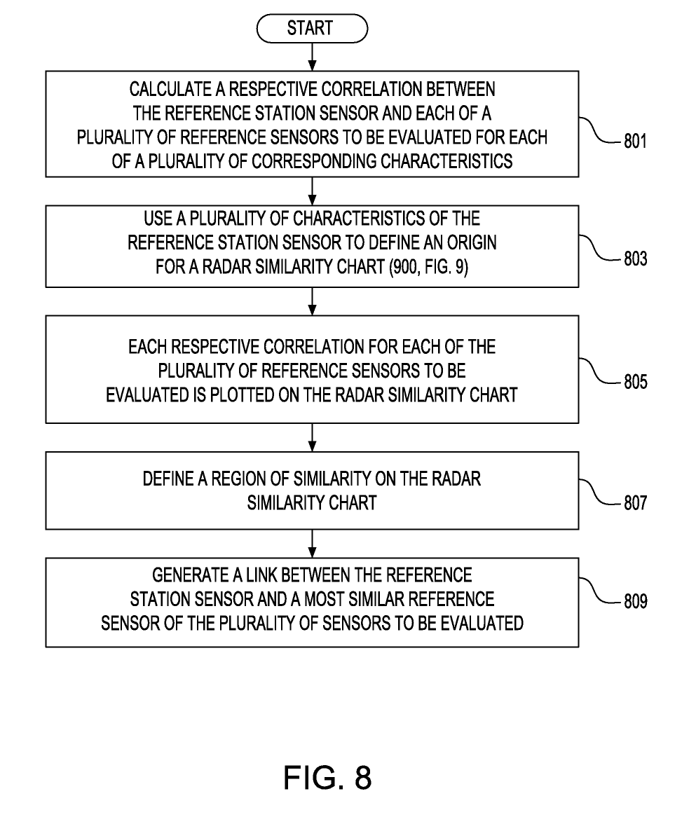

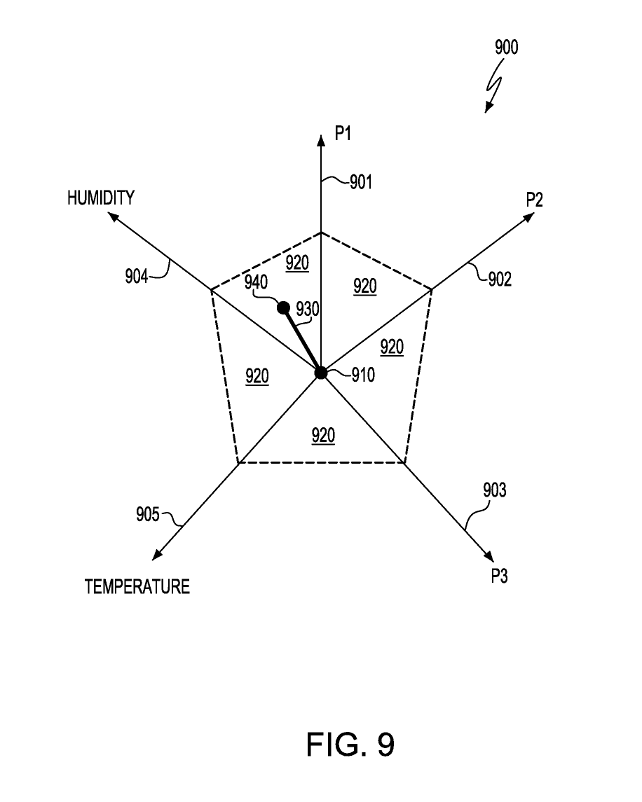

FIG. 8 illustrates an exemplary computer-implemented method for constructing an inheritance network model in accordance with the procedure of block 113 of FIG. 1. FIG. 8 will be discussed in conjunction with FIG. 9 which illustrates an exemplary radar similarity chart 900 for use with the procedure of FIG. 8. The procedure of FIG. 8 commences at block 801 where a respective correlation is calculated between the reference station sensor and each of a plurality of reference sensors to be evaluated for each of a plurality of corresponding characteristics. At block 803, a plurality of characteristics of the reference station sensor are used to define an origin 910 (FIG. 9) for the radar similarity chart 900. The radar similarity chart 900 includes a plurality of respective axes each being associated with a corresponding sensor characteristic. For example, a first axis 901 is associated with a first characteristic P1, a second axis 902 is associated with a second characteristic P2, a third axis 903 is associated with a third characteristic P3, a fourth axis 904 is associated with a humidity characteristic, and a fifth axis 905 is associated with a temperature characteristic.

Next, at block 805 (FIG. 8), each respective correlation for each of the plurality of reference sensors to be evaluated is plotted on the radar similarity chart 900 (FIG. 9). The method progresses to block 807 (FIG. 8) where a region of similarity 920 (FIG. 9) is defined on the radar similarity chart 900. Then, at block 809 (FIG. 8), a link 930 is determined between the reference station sensor and a most similar reference sensor 940 of the plurality of reference sensors to be evaluated.

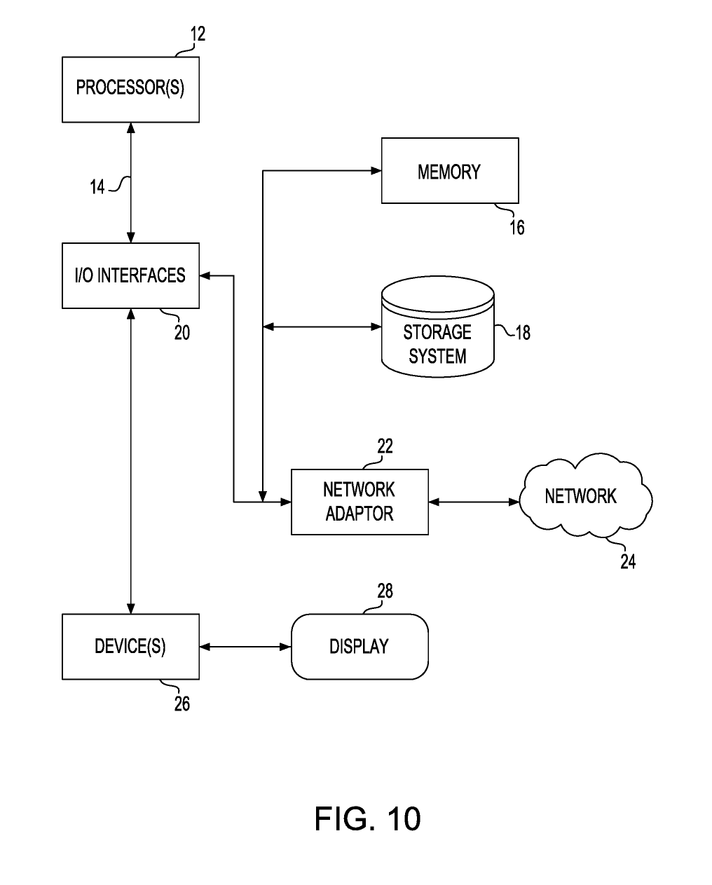

FIG. 10 illustrates an exemplary apparatus in accordance with one or more embodiments of the present invention. This apparatus is only one example of a suitable processing system and is not intended to suggest any limitation as to the scope of use or functionality of embodiments of the present invention. The apparatus/processing system may be operational with numerous other general-purpose or special-purpose computing system environments or configurations. Examples of well-known computing systems, environments, and/or configurations that may be suitable for use with the processing system shown in FIG. 10 may include, but are not limited to, personal computer systems, server computer systems, thin clients, thick clients, handheld or laptop devices, multiprocessor systems, microprocessor-based systems, neural networks, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of the computer system may include, but are not limited to, one or more processors or processing units 12, a system memory 16, and a bus 14 that couples various system components including system memory 16 to processor 12. The processor 12 may execute one or more modules that perform one or more methods in accordance with the present invention, e.g., the exemplary methods described with reference to FIGS. 1, 2, 3, 5 and/or 8. By way of further example, the module(s) may be implemented by the integrated circuits of processor 12, and/or loaded (in the form of processor-readable/executable program instructions) from system memory 16, storage device 18, network 24 or combinations thereof.

Bus 14 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The computer system may include a variety of computer system readable media. Such media may be any available media that is accessible by computer system, and it may include both volatile and non-volatile media, removable and non-removable media.

Memory 16 can include computer system readable media in the form of volatile memory, such as random access memory (RAM, sometimes referred to as system memory), cache memory and/or other forms. Computer system may also include other removable/non-removable, volatile/non-volatile computer system storage media. By way of example only, storage system 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 14 by one or more data media interfaces.

The computer system may also communicate with one or more external devices 26 such as a keyboard, a pointing device, a display 28, etc.; one or more devices that enable a user to interact with the computer system; and/or any devices (e.g., network card, modem, etc.) that enable the computer system to communicate with one or more other computing devices. Such communication can occur via Input/Output (I/O) interfaces 20.

Still yet, the computer system can communicate with one or more networks 24 such as a local area network (LAN), a general wide area network (WAN), and/or a public network (e.g., the Internet) via network adapter 22. As depicted, network adapter 22 communicates with the other components of computer system via bus 14. It should be understood that although not shown, other hardware and/or software components could be used in conjunction with the computer system. Examples include, but are not limited to: microcode, device drivers, redundant processing units, external disk drive arrays, RAID systems, tape drives, and data archival storage systems, etc.

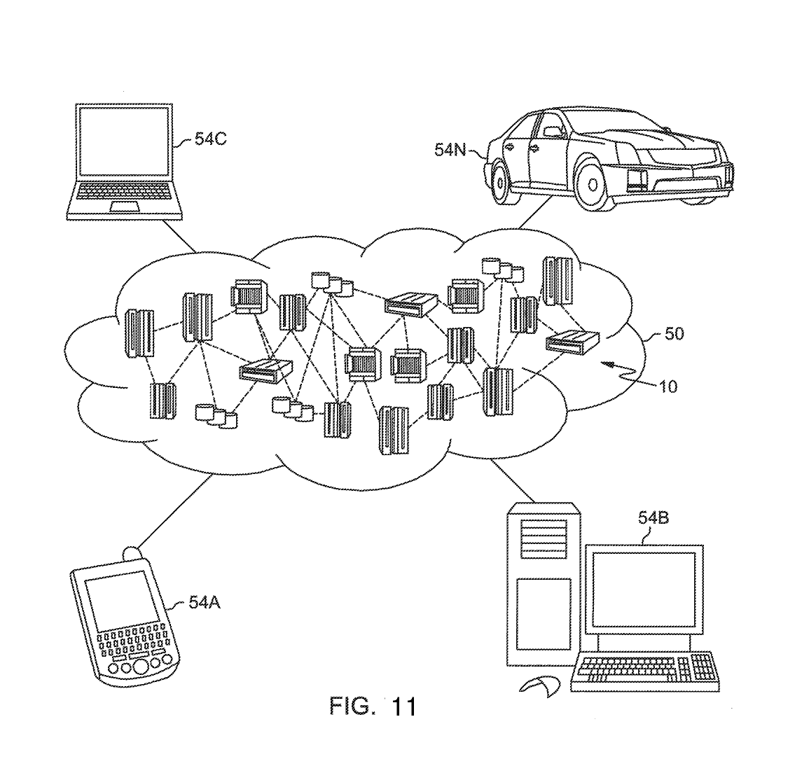

Referring now to FIG. 11, an illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 11 are intended to be illustrative only and that computing nodes 10 and cloud computing environment 50 can communicate with any type of computerized device over any type of network and/or network addressable connection (e.g., using a web browser).

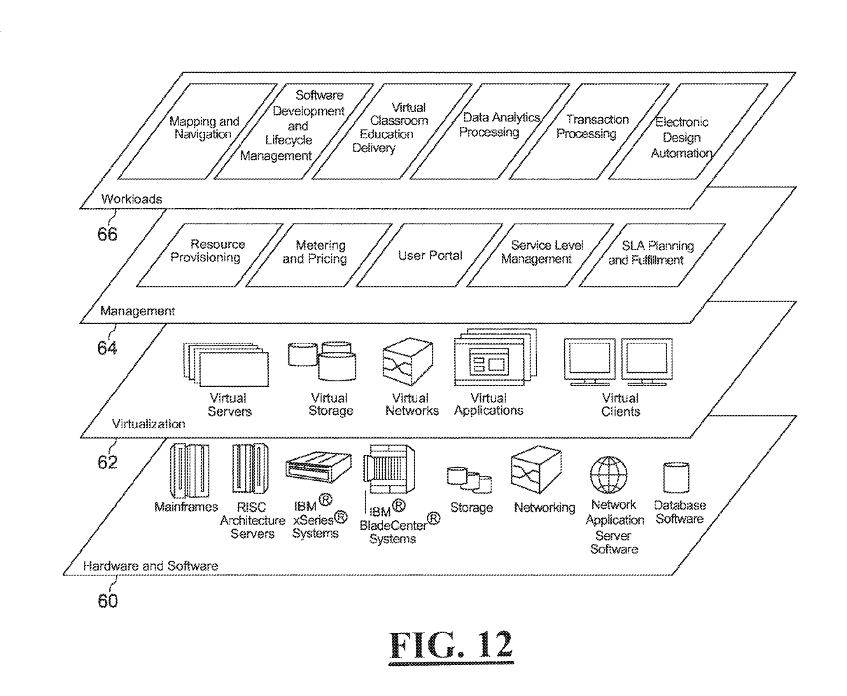

Referring now to FIG. 12, a set of functional abstraction layers provided by cloud computing environment 50 (FIG. 11) is shown. It should be understood in advance that the components, layers, and functions shown in FIG. 12 are intended to be illustrative only and embodiments of the invention are not limited thereto. As depicted, the following layers and corresponding functions are provided:

Hardware and software layer 60 includes hardware and software components. Examples of hardware components include mainframes, in one example IBM zSeries® systems; RISC (Reduced Instruction Set Computer) architecture based servers, in one example IBM pSeries® systems; IBM xSeries® systems; IBM BladeCenter® systems; storage devices; networks and networking components. Examples of software components include network application server software, in one example IBM WebSphere® application server software; and database software, in one example IBM DB2® database software. (IBM, zSeries, pSeries, xSeries, BladeCenter, WebSphere, and DB2 are trademarks of International Business Machines Corporation registered in many jurisdictions worldwide).

Virtualization layer 62 provides an abstraction layer from which the following examples of virtual entities may be provided: virtual servers; virtual storage; virtual networks, including virtual private networks; virtual applications and operating systems; and virtual clients.

In one example, management layer 64 may provide the functions described below. Resource provisioning provides dynamic procurement of computing resources and other resources that are utilized to perform tasks within the cloud computing environment. Metering and Pricing provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal provides access to the cloud computing environment for consumers and system administrators. Service level management provides cloud computing resource allocation and management such that required service levels are met. Service Level Agreement (SLA) planning and fulfillment provide pre-arrangement for, and procurement of, cloud computing resources for which a future requirement is anticipated in accordance with an SLA.

Workloads layer 66 provides examples of functionality for which the cloud computing environment may be utilized. Examples of workloads and functions which may be provided from this layer include: mapping and navigation; software development and lifecycle management; virtual classroom education delivery; data analytics processing; transaction processing; and electronic design automation (EDA).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method for determining a particle concentration, the method comprising:
acquiring, by a computer, a particulate concentration measurement from each of a plurality of sensors to be evaluated at a reference station;
acquiring, by the computer, a reference particulate concentration measurement from a reference station sensor at the reference station;
correlating and selecting, by the computer, a base set of sensors from the plurality of sensors based upon at least one correlation with the reference station sensor;
selecting, by the computer, a reference sensor from the base set of sensors;
constructing, by the computer, a particle concentration calculation model that specifies a relationship between the reference sensor and the reference station sensor;
formulating, by the computer, a generalized sensor type based on one or more characteristics of the reference sensor;
constructing, by the computer, an inheritance network model, based on the generalized sensor type; and
determining, by the computer, a particle concentration based upon the inheritance network model.

2. The computer-implemented method of claim 1 wherein the correlating and selecting, by the computer, of the base set of sensors from the plurality of sensors further comprises calculating a parallel index for each of the plurality of sensors.

3. The computer-implemented method of claim 1 wherein the selecting, by the computer, of the reference sensor from the base set of sensors further comprises generating a set of particle concentration models for each sensor in the base set of sensors.

4. The computer-implemented method of claim 3 wherein the selecting by the computer of the reference sensor from the base set of sensors further comprises obtaining, by the computer, a set of respective reference values from the reference station sensor for each of a corresponding plurality of pollutants.

5. The computer-implemented method of claim 4 wherein the selecting by the computer, of the reference sensor from the base set of sensors further comprises calculating, by the computer, a set of respective particle concentrations for each sensor in the base set of sensors using each respective set of particle concentration models.

6. The computer-implemented method of claim 5 wherein the selecting by the computer, of the reference sensor from the base set of sensors further comprises calculating by the computer, an error level for each respective set of particle concentration models as applied to each sensor in the base set of sensors to generate a set of error levels.

7. The computer-implemented method of claim 6 wherein the selecting by the computer, of the reference sensor from the base set of sensors further comprises selecting as the reference sensor a first sensor of the base set of sensors having a lower error level than a second sensor of the base set of sensors.

8. The computer-implemented method of claim 1, wherein the method is provided as a service in a cloud environment.

9. A computer program product for determining a particle concentration, the computer program product comprising a computer-readable storage medium having a computer-readable program stored therein, wherein the computer-readable program, when executed on a computing device including at least one processor, causes the at least one processor to:
acquire a particulate concentration measurement from each of a plurality of sensors to be evaluated at a reference station;
acquire a reference particulate concentration measurement from a reference station sensor at the reference station;
correlate and select a base set of sensors from the plurality of sensors based upon at least one correlation with the reference station sensor;
select a reference sensor from the base set of sensors;
construct a particle concentration calculation model that specifies a relationship between the reference sensor and the reference station sensor;
formulate a generalized sensor type based on one or more characteristics of the reference sensor;
construct an inheritance network model, based on the generalized sensor type; and
determine a particle concentration based upon the inheritance network model.

10. The computer program product of claim 9 wherein the correlating and selecting of the base set of sensors from the plurality of sensors further comprises calculating a parallel index for each of the plurality of sensors.

11. The computer program product of claim 9 wherein the selecting of the reference sensor from the base set of sensors further comprises generating a respective set of particle concentration models for each sensor in the base set of sensors.

12. The computer program product of claim 11 wherein the selecting of the reference sensor from the base set of sensors further comprises obtaining a set of respective reference values from the reference station sensor for each of a corresponding plurality of pollutants.

13. The computer program product of claim 12 wherein the selecting of the reference sensor from the base set of sensors further comprises calculating a set of respective particle concentrations for each sensor in the base set of sensors using each respective set of particle concentration models.

14. The computer program product of claim 13 wherein the selecting of the reference sensor from the base set of sensors further comprises calculating an error level for each respective set of particle concentration models as applied to each sensor in the base set of sensors to generate a set of error levels.

15. The computer program product of claim 14 wherein the selecting of the reference sensor from the base set of sensors further comprises selecting as the reference sensor a first sensor of the base set of sensors having a lower error level than a second sensor of the base set of sensors.

16. An apparatus for determining a particle concentration, the apparatus comprising:
at least one processor; and
a memory coupled to the at least one processor, wherein the memory comprises program instructions which, when executed by the at least one processor, cause the at least one processor to:
acquire a particulate concentration measurement from each of a plurality of sensors to be evaluated at a reference station;
acquire a reference particulate concentration measurement from a reference station sensor at the reference station;
correlate and select a base set of sensors from the plurality of sensors based upon at least one correlation with the reference station sensor;
select a reference sensor from the base set of sensors;
construct a particle concentration calculation model that specifies a relationship between the reference sensor and the reference station sensor;
formulate a generalized sensor type based on one or more characteristics of the reference sensor;
construct an inheritance network model, based on the generalized sensor type; and
determine a particle concentration based upon the inheritance network model.

17. The apparatus of claim 16 wherein the selecting of the base set of sensors from the plurality of sensors further comprises calculating a parallel index for each of the plurality of sensors.

18. The apparatus of claim 16 wherein the selecting of the reference sensor from the base set of sensors further comprises generating a respective set of particle concentration models for each sensor in the base set of sensors.

19. The apparatus of claim 18 wherein the selecting of the reference sensor from the base set of sensors further comprises obtaining a set of respective reference values from the reference station sensor for each of a corresponding plurality of pollutants.

20. The apparatus of claim 19 wherein the selecting of the reference sensor from the base set of sensors further comprises calculating a set of respective particle concentrations for each sensor in the base set of sensors using each respective set of particle concentration models.
